(19) United States
(12) Patent Application Publication
(10) Pub. No.: US 2024/0033088 A1
(43) Pub. Date: Feb. 1, 2024

(54) Method for Producing a Covering Device for a Bone Defect; Covering Device for a Bone Defect; Method for at Least Partially Covering a Bone Defect by Means of a Bone Defect Covering Device Brought for this Purpose to an Insertion Position

(71) Applicant: ReOss GmbH, Filderstadt (DE)

(72) Inventor: Marcus Seiler, Stuttgart (DE)

(21) Appl. No.: 17/618,549

(22) PCT Filed: Jun. 29, 2020
(86) PCT No.: PCT/DE2020/100562

§ 371 Date: Dec. 13, 2021

(30) Foreign Application Priority Data
Jul. 4, 2019 (DE) 10 2019 118 134.8

(51) Int. Cl. A61F 2/28; A61F 2/30

(52) U.S. Cl. CPC A61F 2/2846; A61F 2/30942; A61F 2002/285; A61F 2002/30985

(57) ABSTRACT

A method for producing a covering device (1) for a bone defect site (2), a covering device (1) for a bone defect site (2) and a method for the at least partial covering of a bone defect site (2) by means of a covering device (1), brought for this purpose into an insertion position, for a bone defect site (2) are proposed, wherein the covering device (1) has a top piece (4), by which the bone defect site (2) is able to be at least partially covered, and at least one fixing means (5) for fixing the top piece (4) on the bone, wherein on the top piece (4) at last one fixing means (5), suitable for fixing the top piece (4) is detachably arranged by means of at least one connection (12).

### CROSS REFERENCE TO RELATED APPLICATIONS

This application is the U.S. national stage of International Application No. PCT/DE2020/100562, filed on 2020 Jun. 29. The international application claims the priority of DE 102019118134.8 filed on 2019 Jul. 4; all applications are incorporated by reference herein in their entirety.

### BACKGROUND

The invention proceeds from a method for producing a covering device for a bone defect site, according to the type of claim 1, a covering device for a bone defect site according to the type of claim 15, and a method for at least partially covering a bone defect site by means of a bone defect site covering device brought for this purpose into an insertion position, according to the type of claim 28.

Bone defect sites in the form of recesses or cavities in endogenous bone tissue are often filled with bone reconstruction material in bone surgery, for example in the reconstruction of bone in orthopaedic, neurosurgical or plastic surgery or in maxillofacial operations. The bone reconstruction material generally consists of a mixture of synthetic bone substitute material (e.g. hydroxyl apatite granulate) and endogenous bone particles. So that the bone reconstruction material is substantially grown through with bone exclusively from the bone side, the recess, as described in patent document DE 43 02 708 C2, is closed by a covering membrane. The covering membrane is fastened by fastening nails on the endogenous healthy bone, adjoining the bone defect site, which healthy bone is damaged by the fastening nails, wherein, as the covering membrane consists of a flexible material, the fastening requires a highest level of manual skill by the surgeon.

In order to overcome this disadvantage of a lacking support function of the covering membrane, a covering membrane is described in patent document US 48 16 339 which consists of several layers, wherein these layers do not consist of resorbable membrane material. It is necessary here, if applicable, that after the healing of the bone defect, a second intervention is necessary in order to remove material which is foreign to the body.

In patent document DE 10 2005 039 382 B4, a biodegradable hollow body, which in particular has a hollow-cylindrical or conical-cylindrical shape, is proposed. The hollow body has in its walls a plurality of openings through which an uptake of blood and thereby the building up of autogenous bone is possible. A disadvantage here is that, for the insertion of the hollow body, a cylindrical bore must be introduced into the existing bone by means of a drill.

In the published patent application DE 10 2006 047 054 A1 an implant bed is proposed which is distinguished by a high fitting accuracy and stability, so that the attending doctor can easily handle it and implant it. The implant bed, made from hydroxl apatite, which has, on the side facing the mucous membrane, a thin membrane consisting in particular resorbable material for protection of the mucous membrane from mechanical effects and for protection of the implant bed from ingrowing tissue from the mucous membrane, is produced by a building-up production method so that the material property has a “gradient structure” in the sense of an in particular inwardly decreasing density. Here, on the side facing the bone, a construction is provided having an in particular porous structure, and on the outer side of the implant bed, on which a structure is situated for mounting a dental implant and/or a denture, a compact construction is provided.

Furthermore in the published patent applications DE 198 30 992 A1, DE 10 2005 060 761 A1, DE 42 26 465 A1, WO 01/91818 A1, DE 10 2005 041 412 A1, DE 10 2006 047 054 A1, US 2011/0151400 A1, WO 00/59409 A1, WO 96/12446 A1, EP 2 737 871 A2 and WO 2006/051401 A2, as well as the utility model DE 20 2015 005 610 U1 and the patent document U.S. Pat. No. 7,172,422 B1 devices are described for a bone defect site, wherein all of these solutions have the disadvantage they affect the healthy bone which is present adjacent to the bone defect site.

In order to prevent this disadvantage, in the published patent applications DE 10 2011 011 191 A1 and DE 10 2016 000 236 A1 a precisely fitting covering device is proposed which, however, has the disadvantage, directly through the fitting accuracy, that its positioning on the bone defect site is made difficult.

In order to prevent this disadvantage, in the published patent application DE 10 2015 006 154 A1 a covering device is proposed which has a top piece which has a side facing away from the bone defect and a side facing the bone defect and, if applicable, at least one fixing means for fixing the top piece on a bone, wherein the top piece consists of a dimensionally stable material which is at least partially in contact with the bone, and a side of the top piece facing the bone defect or a side of the top piece facing away from the bone defect corresponds to the shape of the regenerated bone, wherein the top piece has at least one positioning means. The positioning of the top piece is indeed facilitated by the positioning means in an insertion position, however the problem is not thereby solved that the fixing of the top piece by means of a fixing means possibly requires a highest level of manual skill by the surgeon.

### SUMMARY

The method according to the invention for producing a covering device for a bone defect site, wherein the term “bone defect site” designates a site of a bone (diseased, deformed, injured, altered through ageing processes, altered through degeneration (e.g. after dental extraction, tumour etc.) and/or altered in volume) (e.g. hip, spine, head, jaw or suchlike) of a human or animal, which deviates from the shape and/or volume of a healthy bone, having the features of claim 1, the covering device for a bone defect site, having the features of claim 15, and the method according to the invention for the at least partial covering of a bone defect site by means of a bone defect covering site brought for this purpose into an insertion position, having the features of claim 29, have by comparison the advantage that the covering device consists of a top piece (e.g. mould shell, rigid shell, shaped body), which can be configured having one or several layers, and at least one fixing means for fixing the top piece on a bone, wherein on the top piece at least one fixing means suitable for the fixing of the top piece is detachably arranged by means of a connection, whereby an insertion of the covering device is greatly facilitated.

### DETAILED DESCRIPTION

The method according to the invention for producing a covering device for a bone defect site, which has a top piece and at least one fixing means for the top piece, consists of the following method steps:
Recording of a first dataset, which represents the affected bone detect site in the actual state,
Comparison of the first dataset with a second dataset, which represents the target state of a regenerated bone at the bone defect site,
Use of the first dataset and of the second dataset for planning the top piece,
   which is suitable to at least partially cover the bone defect site, wherein the top piece has a side facing away from the bone defect site and a side facing the bone defect site, and the top piece is able to be fixed by at least one fixing means within the bone defect site,
or
   which is suitable to at least partially cover the bone defect site and, in addition, to at least partially cover a healthy bone adjoining the bone defect site, wherein the top piece has a side facing away from the healthy bone and a side facing away from the bone defect site, and a side facing the healthy bone and a side facing the bone defect site, and the top piece is able to be fixed by at least one fixing means within the bone defect site and/or by at least one fixing means on the healthy bone,
Implementation of the planning of the top piece into a planning dataset and
Delivery of the planning dataset to a manufacturing method, in particular to a computer-controlled manufacturing method, wherein during and/or after the manufacture of the top piece (4) at least one fixing means suitable for fixing the top piece within the bone defect site or on the healthy bone adjoining the bone defect site is detachably connected to the top piece by means of at least one connection arranged between the fixing means and the top piece, wherein the detaching of at least one connection is irreversible and at least one connection is a predetermined breaking point.

In the production of the covering device, preferably a computer-aided design (CAD) of the top piece is combined with a computer-aided manufacture (CAM) to CAD/CAM, so that a design model of the top piece, developed on computer, can be electronically transferred directly to the manufacture.

According to an advantageous embodiment of the method according to the invention, during at least one preceding method step for at least one fixing means, a fixing means position, adapted specifically to the use of this fixing means, which fixing position enables a fixing of the top piece within the bone defect site or on the healthy bone, is predetermined in order to obtain through the manufacturing method a top piece on which at least one fixing means is detachably connected to the top piece in its fixing means position by means of at least one connection.

According to an advantageous embodiment of the method according to the invention in this respect, in the manufacturing method at at least one fixing means position which is adapted for a fixing means, a guide is arranged for a tool, by which the creating of a bore, suitable for the fixing means, is enabled within the bone defect site or on the healthy bone.

According to an additional advantageous embodiment of the method according to the invention, the side of the top piece, formed in the manufacturing method, facing the bone defect or the side facing away from the bone defect, corresponds at least partially to the shape of the regenerated bone, which through its regeneration if possible has the shape of a healthy bone again, in the target state.

According to an additional advantageous embodiment of the method according to the invention, the second dataset arises through calculation or was recorded at a time at which the bone was still a healthy bone at the bone defect site which is now to be regenerated.

According to an additional advantageous embodiment of the method according to the invention, the second dataset arises through calculation or was recorded at a time at which the bone was still a healthy bone at the bone defect site which is now to be regenerated. In the latter case, it is possible that if applicable the ideal state (target state) of the bone is documented so that it is known how a bone which is possibly to be regenerated later is to appear. In humans, the recording of the dataset of the healthy bone should preferably take place at the age of 18 to 25 years. Of course, it is also conceivable that in fully grown state several healthy bones or the entire skeleton of the human or animal are recorded, documented and/or stored. It would also be conceivable that already at the time of recording the healthy bone, a top piece is at least partially made. Preferably, the recording of the dataset of the healthy bone or respectively the recordings of the healthy bones, take place after the healthy bone is fully grown.

According to an advantageous embodiment of the method according to the invention in this respect, the dataset of the healthy bone or respectively the datasets of the healthy bones are stored (preserved) on a storage medium for later use thereof.

According to an additional advantageous embodiment of the method according to the invention, the recording of the first data set represents the affected bone defect site in its three-dimensionality and/or the recording of the second dataset represents the shape of the still healthy bone in its three-dimensionality.

According to an additional advantageous embodiment of the method according to the invention, the recording of the first dataset, which represents the actual state, and/or the recording of the second dataset, which represents the target state, takes place by at least one imaging method.

According to an additional advantageous embodiment of the method according to the invention, the recording of the first dataset and/or the recording of the second dataset takes place by at least one method which enables a three-dimensional presentation of a bone. In particular, the recording of the first dataset and/or the recording of the second dataset take place by means of tomography, computer tomography, digital volume tomography, sonography or suchlike.

According to an additional advantageous embodiment of the method according to the invention, in the manufacturing method the top piece is formed by 3D printing and/or by means of milling.

According to an additional advantageous embodiment of the method according to the invention, during the manufacture of the top piece at least one fastening device for at least one implant which is to be set and/or at least one predetermined breaking point and/or at least one positioning means are arranged on the top piece. The fastening device can be configured for example as a recess.

According to an advantageous embodiment of the method according to the invention in this respect, at least one fastening device (e.g. recess) is exposed by removing a portion of the top piece which, before removal by means of at least one predetermined breaking point, is connected to the remaining portion of the top piece. The point in time of the exposing of the fastening device can lie here before or after the arranging of the covering device on the bone defect site.

According to an additional advantageous embodiment of the method according to the invention, at least one positioning means, arranged on the top piece during the manufacture of the top piece, serves for the positioning of the top piece in an insertion position. The positioning means is preferably arranged at the edge of the top piece. Preferably at least one positioning means serves for the positioning of the top piece on a healthy bone adjoining the bone defect site, so that this at least one positioning means has a side facing away from the healthy bone and a side facing the healthy bone and at least partially corresponding thereto.

According to an additional advantageous embodiment of the method according to the invention, at least one predetermined breaking point is arranged between the top piece and a positioning means, whereby preferably a positioning means which rests on the healthy bone and is thus possibly bulky in an intrusive manner, e.g. after fixing of the top piece or after the regeneration of the bone at the bone defect site, can be removed from the possibly remaining top piece, and/or at least one predetermined breaking point is arranged between the top piece and at least one fixing means arranged on the top piece.

According to an additional advantageous embodiment of the method according to the invention, a cleaning- and/or sterilisation process is carried out during the manufacture of the top piece.

According to an additional advantageous embodiment of the method according to the invention, the top piece and/or at least one fixing means and/or at least one positioning means are formed from a dimensionally stable, in particular rigid, material.

By the method according to the invention for producing a covering device for a bone defect site, a covering device can be created, the top piece and/or fixing means of which for example of a synthetic material and/or of a material of autogenic, synergenic, allogenic or xenogenic origin, human and/or animal bones or respectively the human, animal or synthetic matrix has a shape by which the region situated between the bone and the desired shape of the regenerated bone is filled entirely or almost entirely. For this, for example a bone block is removed from the donor, said bone block being subsequently modelled if applicable by means of CAD/CAM.

The covering device for a bone defect site according to the invention, which has a top piece by which the bone defect site is able to be at least partially covered, wherein the top piece has a side facing away from the bone defect site and a side facing the bone defect site, or by which the bone defect side is able to be at least partially covered, and in addition a healthy bone adjoining the bone defect site is able to be at least partially covered, wherein the top piece has a side facing away from the healthy bone and a side facing away from the bone defect site and a side facing the healthy bone and a side facing the bone defect site, and a fixing means for fixing the top piece within the bone defect site and/or with at least one fixing means for fixing the top piece on the healthy bone adjoining the bone defect site, on the top piece at least one fixing means, suitable for fixing the top piece within the bone defect site or on the healthy bone adjoining the bone defect site, is arranged detachably by means of at least one connection arranged between the fixing means and the top piece, wherein the detaching of at least one connection is irreversible and at least one connection is a predetermined breaking point.

According to an additional advantageous embodiment of the covering device according to the invention, a fixing means position, adapted specifically for the use of this fixing means is present on the top piece for a fixing means, which fixing means position enables a fixing of the top piece within the bone defect site or on the healthy bone, or on the top piece for at least one fixing means at least one fixing means position, adapted specifically to the use of at least one fixing means, is present, which enables a fixing of the top piece within the bone defect site or on the healthy bone, wherein at least one fixing means is detachably connected to the top piece by means of at least one connection in its fixing means position which is adapted specifically to the use of this fixing means.

According to an additional advantageous embodiment of the covering device according to the invention, the top piece has at least one predetermined breaking point. The predetermined breaking point has the advantage that, in so far as the top piece is to be removed after a successful bone regeneration, this removal can take place in a minimally invasive manner, without “having to expose everything”, because the top piece, owing to the predetermined breaking point, can be broken up into at least two parts. The removal of the top piece (e.g. after a bone regeneration) is therefore very easily possible. Furthermore, the predetermined breaking point can serve for parts of the top piece, which are not or are no longer required, to be separated from the remainder of the top piece.

According to an additional advantageous embodiment of the covering device according to the invention, at least one fixing means, which is preferably detachably connected to the top piece in its fixing means position adapted specifically to the use of this fixing means, projects over the side facing away from the bone defect site or the side of the top piece facing away from the healthy bone and/or the side facing the bone defect site or the side of the top piece facing the healthy bone.

According to an additional advantageous embodiment of the covering device according to the invention, the top piece has a guide for a tool at at least one fixing means position which is adapted to a fixing means. Preferably, through the guide for a tool, the creation of a bore, suitable for the fixing means, within the bone defect site or on the healthy bone is made possible.

According to an additional advantageous embodiment of the covering device according to the invention, the top piece consists of a dimensionally stable, in particular rigid, material.

According to an additional advantageous embodiment of the covering device according to the invention, the side facing the bone defect or the side of the top piece facing away from the bone defect corresponds at least partially to the shape of a regenerated bone at the bone defect site, which through its regeneration has the shape of a healthy bone again.

According to an additional advantageous embodiment of the covering device according to the invention, the material of the top piece is of organic and/or inorganic origin and/or the material of a fixing means is of organic and/or inorganic origin. This can also be an autogenic, syngenic, allogenic, xenogenic, synthetic or alloplastic material.

According to an additional advantageous embodiment of the covering device according to the invention, the top piece and/or at least one fixing means consist at least partially of a biocompatible material.

According to an additional advantageous embodiment of the covering device according to the invention, the top piece and/or at least one fixing means and/or at least one positioning means can consist at least partially of a resorbable material. Advantageously, the resorption time of the rigid shell can be controlled by its resorption gradient and/or the resorption time can also be less than six months, so that the implant can be inserted in a timely manner. Preferably, resorbable metals or alloys, in particular magnesium or magnesium alloys, can be used. The 3D models (e.g. the top piece and/or the fixing means) are preferably built up by the laser melting method under vacuum, wherein preferably a 3D printer is used.

According to an additional advantageous embodiment of the covering device according to the invention, the top piece and/or at least one fixing means and/or at least one positioning means consist at least partially of a biodegradable material.

According to an additional advantageous embodiment of the covering device according to the invention, the top piece and/or at least one fixing means and/or at least one positioning means consist at least partially of a polymer or a polymeric compound.

According to an additional advantageous embodiment of the covering device according to the invention, the top piece and/or at least one fixing means and/or at least one positioning means consist at least partially of a biocompatible material.

According to an additional advantageous embodiment of the covering device according to the invention, the top piece and/or at least one fixing means and/or at least one positioning means consist at least partially of a resorbable or non-resorbable polymer (e.g. polylactide (PLA), polycaprolactone (PCL), bioresorbable alloys (e.g. based on magnesium)).

According to an additional advantageous embodiment of the covering device according to the invention, the top piece and/or a positioning means have a wall thickness of at least 0.1 mm, preferably 0.2-0.3 mm in particular in the case of titanium, and 0.5-1 mm in particular in the case of polycaprolactones, however at least as much so that a dimensional stability of the top piece or respectively of a positioning means results.

According to an additional advantageous embodiment of the covering device according to the invention, the top piece and/or at least one positioning means has a constant or a varying wall thickness.

According to an additional advantageous embodiment of the covering device according to the invention, at least one fixing means is a pin, a screw, a nail and/or a bone adhesive. In order to conserve the healthy bone, the fixing means is or respectively are preferably arranged in the region of the bone defect site.

According to an additional advantageous embodiment of the covering device according to the invention, the top piece has at least one milling (bore for the fixing means).

According to an additional advantageous embodiment of the covering device according to the invention, the filling corresponds to the fixing means.

According to an additional advantageous embodiment of the covering device according to the invention, the wall facing the bone defect has a surface conditioning.

According to an additional advantageous embodiment of the covering device according to the invention, the surface can have a microstructuring, pores, osteoblast attractants, means for promoting bone growth and/or bone substitute means containing BMP.

According to an additional advantageous embodiment of the covering device according to the invention, the top piece has at least one opening. This means that the top piece does not have to have a closed wall. Through a plurality of openings, the top piece can have, at least in places. A network structure, wherein the side of the top piece of the network structure facing away from the bone defect or the side of the top piece of the network structure facing the bone defect corresponds to the shape of the regenerated bone.

According to an additional advantageous embodiment of the covering device according to the invention, the top piece has at least one fastening device (e.g. a recess) for at least one implant which is to be set.

According to an additional advantageous embodiment of the covering device according to the invention, at least one fastening device (e.g. a recess) is covered at least partially by a portion of the top piece which is connected to the remaining portion of the top piece by means of at least one predetermined breaking point.

According to an additional advantageous embodiment of the covering device according to the invention, for positioning the top piece on a healthy bone adjoining the bone defect site, at least one positioning means is arranged on the top piece, which positioning means has a side facing away from the healthy bone and a side facing the healthy bone and at least partially corresponding thereto.

According to an additional advantageous embodiment of the covering device according to the invention, at least one predetermined breaking point is arranged between the top piece and at least one positioning means.

According to an additional advantageous embodiment of the covering device according to the invention, the covering device is produced in accordance with the method for producing a covering device for a bone defect site according to one of claims 1 to 14.

The method according to the invention for the at least partial covering of a bone defect site by means of a covering device, brought for this purpose into an insertion position, which has a top piece and at least one fixing means for the top piece,
wherein the top piece is suitable to at least partially cover the bone defect site, wherein the top piece has a side facing away from the bone defect site and a side facing the bone defect site, and the top piece is able to be fixed by at least one fixing means within the bone defect site, or
wherein the top piece is suitable to at least partially cover the bone defect site and in addition to at least partially cover a healthy bone adjoining the bone defect site, wherein the top piece has a side facing away from the healthy bone and a side facing away from the bone defect site and a side facing the healthy bone and a side facing the bone defect site, and the top piece is able to be fixed by at least one fixing means within the bone defect site and/or by at least one fixing means on the healthy bone, at least one fixing means, suitable for fixing the top piece, is detachably arranged on the top piece by means of at least one connection, wherein a connection between the fixing means and the top piece is detached, wherein the detaching of at least one connection is irreversible and at least one connection is a predetermined breaking point, whereby the top piece is able to be fixed by means of this fixing means within the bone defect site or on the healthy bone.

According to an advantageous embodiment of the method according to the invention, on the top piece for at least one fixing means at least one fixing means position, adapted specifically to the use of at least one fixing means, which position enables a fixing of the top piece within the bone defect site or on the healthy bone, is predetermined, wherein at least one fixing means in its fixing means position, adapted specifically to the use of this fixing means, which enables a fixing of the top piece within the bone defect site or on the healthy bone, is detachably connected to the top piece so that for fixing the top piece within the bone defect site or on the healthy bone by means of this fixing means, detachably connected to the top piece, the connection between the fixing means and the top piece is detached, whereby the top piece by means of this fixing means is able to be fixed within the bone defect site or on the healthy bone.

According to an additional advantageous embodiment of the method according to the invention, the fixing means is detachably connected to the top piece by means of at least one predetermined breaking point, so that the detaching of the connection between the fixing means and the top piece takes place by breaking of the at least one predetermined breaking point.

According to an additional advantageous embodiment of the method according to the invention, after the detaching of the fixing means from the top piece, the top piece is immediately able to be fixed by means of this fixing means, or after the detaching of the fixing means from the top piece, the fixing means is removed from the fixing means position, in order to carry out at least one method step before a fixing of the top piece, possible thereby, by means of this fixing means which is brought into the fixing means position again.

According to an additional advantageous embodiment of the method according to the invention, at least one positioning means is arranged on the top piece, wherein at least one positioning means is used in order to bring the covering device into the insertion position. Preferably at least one positioning means is removed from the top piece after the covering device has been brought into the insertion position. By the at least one positioning means, preferably a positioning of the top piece on a healthy bone adjoining the bone defect site is possible, because the positioning means has a side facing away from the healthy bone and a side facing the healthy bone and at least partially corresponding thereto. The top piece is preferably arranged and/or fixed here exclusively in the region of the bone defect site, which is covered entirely or at least partially by the top piece, so that it does not affect the healthy bone adjoining the bone defect site, at which healthy bone, due to its health, in any case no regeneration of the bone takes place. Instead of the only partial covering of the bone defect site by the top piece, the top piece is therefore preferably adapted to the bone defect site in an accurately fitting manner and preferably terminates flush with the healthy bone. Hereby, the bone defect site is entirely covered by the top piece, which preferably is not projecting beyond the bone defect site.

According to an additional advantageous embodiment of the method according to the invention, as a covering device a covering device is used which was produced in accordance with the method for producing a covering device for a bone defect site according to one of claims 1 to 14, and/or as a covering device a covering device is used which is a covering device according to one of claims 15 to 27.

Further advantages and advantageous embodiments of the invention are able to be seen from the following description, the drawings and the claims.

### BRIEF DESCRIPTION OF THE DRAWINGS

Example embodiments of the subject of the invention are illustrated in the drawings and are explained more closely below. There are shown:

FIG. 1 a perspective illustration of a covering device for a bone defect site according to the invention,

FIG. 2 another perspective illustration of the covering device for a bone defect site according to the invention, in accordance with FIG. 1,

FIG. 3 another perspective illustration of the covering device according to the invention for a bone defect site, in accordance with FIG. 1,

FIG. 4 a top view onto the covering device according to the invention for a bone defect site, in accordance with FIG. 1, and

FIG. 5 a detail view of the covering device according to the invention for a bone defect site, in accordance with FIG. 1.

### DETAILED DESCRIPTION OF THE PREFERRED EMBODIMENTS

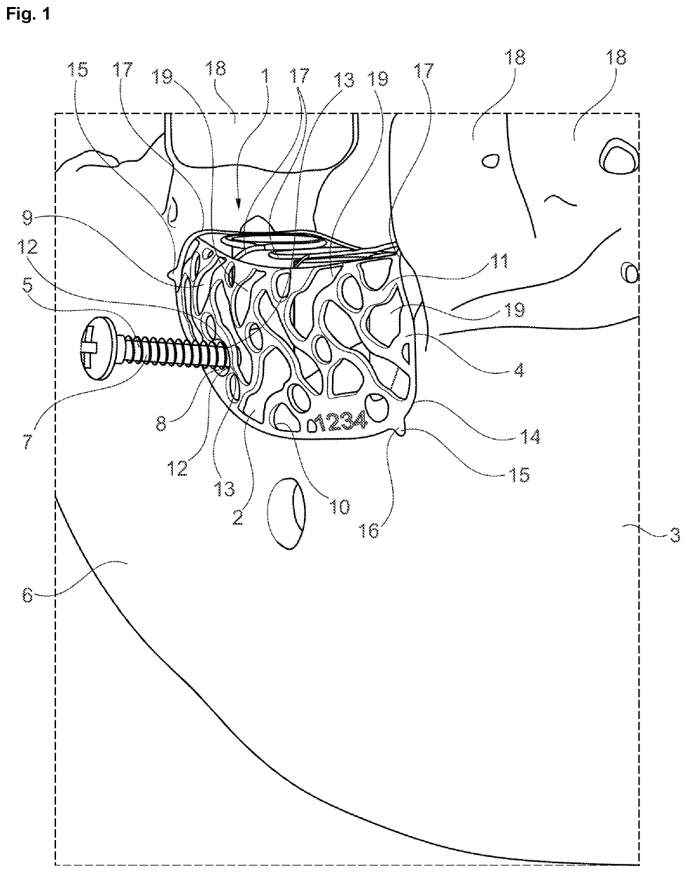

FIG. 1 shows a perspective illustration of a covering device 1 according to the invention for a bone defect site 2 (bone defect) of a bone, in particular of a jawbone 3. The covering device 1 according to the invention, which serves for the covering and/or reconstruction of a bone defect site 2, consists of a top piece 4, which is single-layered, and of a fixing means 5, which is illustrated in FIG. 1 as a screw and which is able to be arranged in the bone defect site 2, in order not to injure the healthy bone adjoining the bone defect site 2. Of course, it is also conceivable that several fixing means 5 are used for fixing the top piece 4, wherein these would preferably also be arranged in the bone defect site 2. The top piece 4 is made from a dimensionally stable, in particular rigid, material, so that it is self-supporting and no additional support is necessary. For fixing the top piece 4 (mould shell, rigid shell), the fixing means 5 is screwed through a bore 6 into the jawbone 3. For this, preferably a bore, which is not illustrated, is produced in the jawbone 3. It is also conceivable that instead of the screw 7, a pin or a pin with at least one barb is used as fixing means 5. In this case, the subsequent fixing of the top piece 4 preferably takes place by means of ultrasonic welding. In ultrasonic welding, preferably an ultrasonic generator generates a precisely defined frequency, which is concentrated via a sonotrode. After the placing of the resorbable fixing means 5 (pin) onto a borehole (bore) which is pre-bored in the bone, a generated vibration provides for a liquefication of the pin surfaces at the edges thereof, whereby a sliding of the pin into the borehole is brought about. Through the change of the physical state, the pin also advances into the bony cavities which are unable to be reached by a conventional bone screw (screw 7), so that a high initial strength is achieved. In addition, the pin head connects itself with the top piece 4 and, with a blocking mechanism, provides for a stable three-dimensional construct. With the ultrasonic welding, the fixing means 5 is thus softened, so that it connects with the jawbone 3 and with the top piece 4.

Through the fixed top piece 4, a sealed interior 9 is produced between the jawbone 3 and the top piece 4, which is filled through the regeneration of the bone and/or through introduction of a material or organic and/or inorganic origin, which can also be an autogenic, syngenic, allogenic, xenogenic, synthetic and/or alloplastic material, so that the regenerated bone or the introduced material corresponds to the shape of the side of the single-layered top piece 4 facing the bone defect site 2. It would also be conceivable that the side 11 of the single-layered top piece 4, facing away from the bone defect site 2, corresponds to that of the shape of the bone defect site 2. In order to accelerate the regeneration process of the jawbone 3, the side of the top piece 4 facing the bone defect can have a surface conditioning (e.g. a microstructuring, pores, osteoblast attractants, means for promoting bone growth and/or bone substitute means containing BMP).

In order to facilitate the handling of the fixing means 5 and/or in order to ensure that the fixing means 5 is arranged on the top piece at its planned fixing position, the fixing means 5 is detachably connected to the top piece 4 by at least one connection 12. This means that the fixing means 5 is already connected to the top piece 4 before the insertion of the top piece 4 into the bone defect site. FIG. 1 shows the covering device 1 after its insertion into the bone defect site 2, wherein the covering device 1 is not yet fixed to the bone defect site 2 by the fixing means 5. With the use of the fixing means the connection 12 is separated irreversibly (irrevocably) for example by pressure onto the fixing means 5 and/or a twisting of the fixing means 5 so that the fixing means can no longer be connected to the top piece 4 by means of the connection 12. The fixing means 5 is preferably connected to the top piece 4 by means of at least one connection 12, which can be realized e.g. by at least one web, which functions as a predetermined breaking point 13. After the detaching of the connection 12, therefore e.g. by breaking the web or respectively the predetermined breaking point 13, the fixing means 5 can be used as intended. It is also conceivable that several fixing means 5 are used, wherein at least one fixing means 5 is detachably connected to the top piece by means of at least one connection 12, wherein the detaching of the connection 12 is irreversible, so that at least one fixing means 5 can no longer be connected to the top piece 4 by means of the connection 12.

At the edge 14 of the top piece 4 are positioning means 15, which have a side facing a healthy bone 6 and a side 16 facing away from the healthy bone 6. In the arrangement of the top piece 4 as intended at the bone defect site 2, which is supported by the positioning means 15, the side facing a healthy bone at least partially touches the healthy bone 6, whereby through the positioning means 15 a problem-free seat of the top piece 4 is guaranteed, if applicable even without arrangement of at least one fixing means 5 or at least until at least one fixing means 5 is arranged in the bone defect site 2. In order to be able to easily remove the top piece 4 after the bone regeneration in a minimally invasive manner, this has predetermined breaking points 17, whereby after its separation it can be divided e.g. into two parts for its removal.

FIG. 1 shows the top piece 4 arranged at the bone defect site 2 of the partially illustrated jawbone 3, which has teeth 18. Hereby it becomes apparent that the top piece 4 is preferably arranged only in the region of the bone defect site 2 of the jawbone 3, so that it neither spans nor touches a healthy bone 6. Thus only the positioning means 15, arranged on the top piece 4, have contact with the healthy bone 19. It is conceivable that between the positioning means 15 and the top piece 4 at least one predetermined breaking point, not illustrated, is arranged, whereby the positioning means 15, after the fixing of the top piece 4 in the bone defect site 2, can be separated from the top piece 4.

In FIG. 1 a top piece 4 is illustrated, the side 10 of which, facing the bone defect, corresponds to the shape of the regenerated bone. It is also conceivable that the positioning means 15 are arranged on the top piece 4 such that the side 11 of the top piece 4 facing away from the bone defect corresponds to the shape of the regenerated bone. This could be brought about e.g. by arranging the positioning means 15 on the side 11 of the top piece 4 facing away from the bone defect.

The regions 19, which represent the position of the implants which are to be inserted in future, are illustrated schematically.

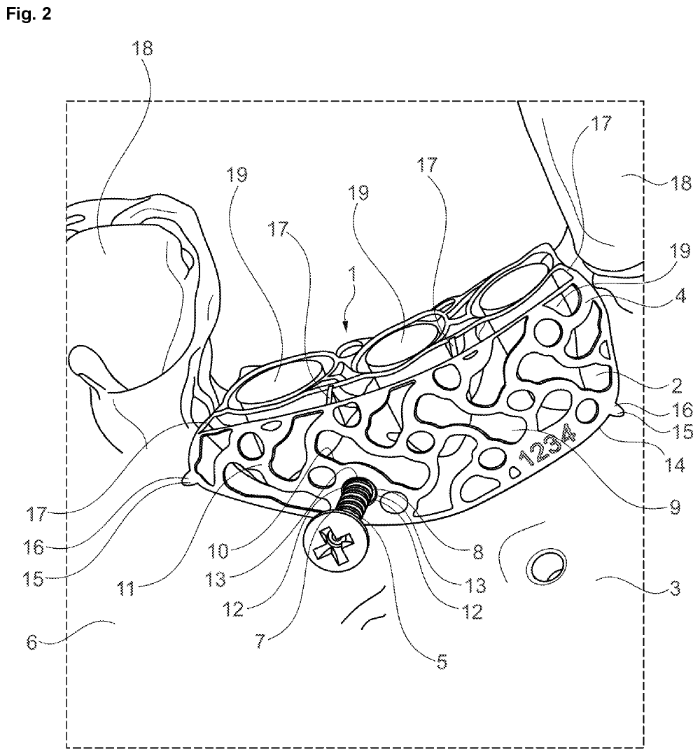

FIG. 2 shows another perspective illustration of the covering device 1 according to the invention for a bone defect site 1, according to FIG. 1.

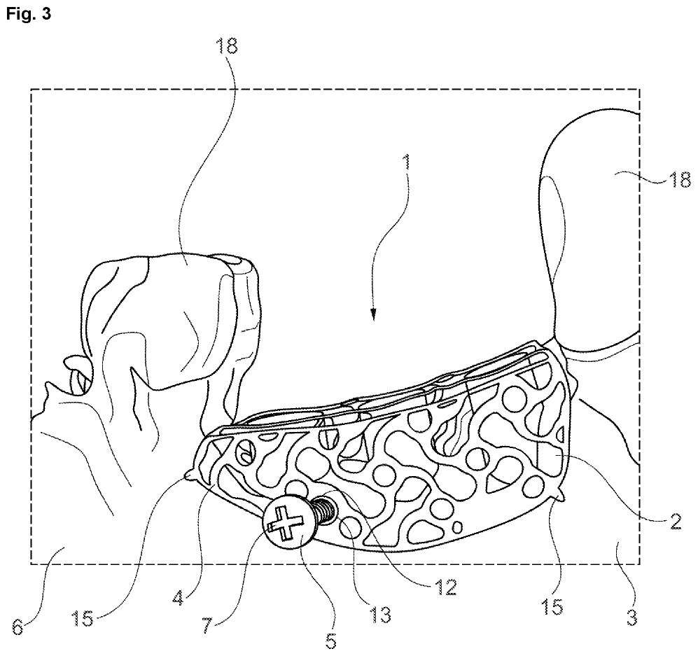

FIG. 3 shows another perspective illustration of the covering device 1 according to the invention for a bone defect site 2, according to FIG. 1.

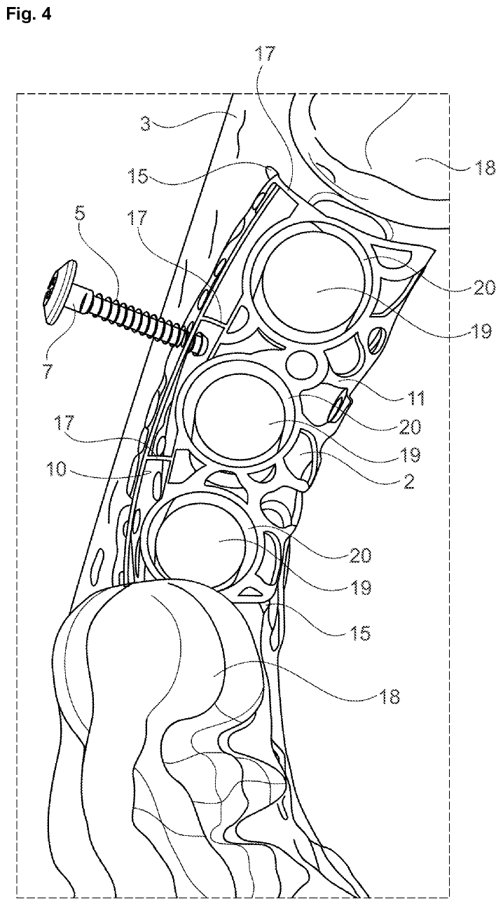

FIG. 4 shows a top view onto the covering device 1 according to the invention for a bone defect site 2, according to FIG. 1. For the future inserting of the implants, the top piece 4 has openings 20.

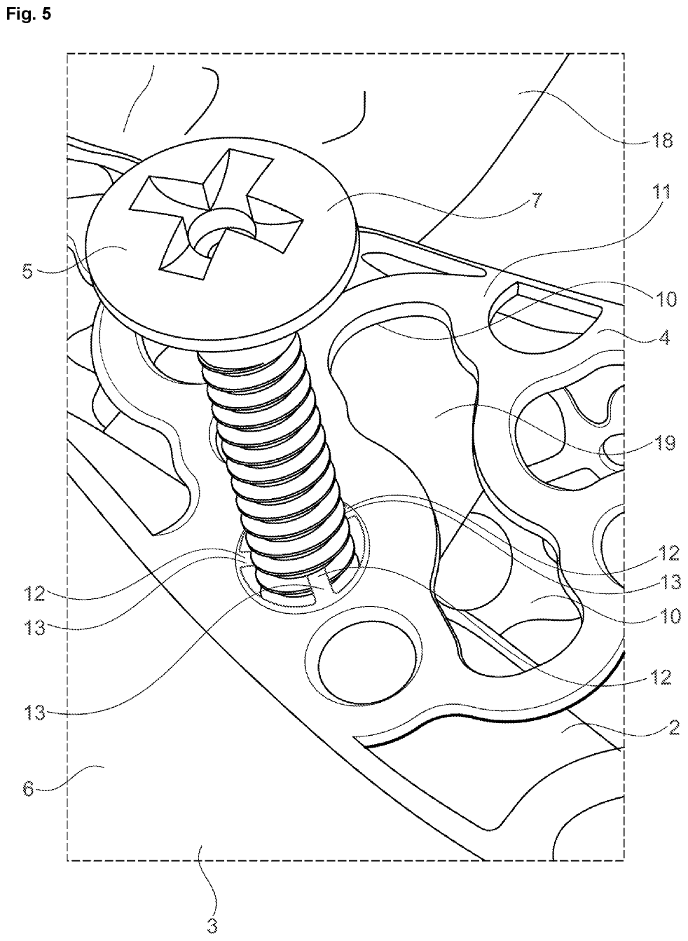

FIG. 5 shows a detail view of the covering device 1 according to the invention for a bone defect site 2, according to FIG. 1.

All the features illustrated here can be essential to the invention both individually and also in any desired combination with one another.

### LIST OF REFERENCE NUMERALS

1 covering device
2 bone defect site
3 jawbone
4 top piece
5 fixing means
6 healthy bone
7 screw
8 bore
9 interior
10 side
11 side
12 connection
13 predetermined breaking point
14 edge
15 positioning means
16 side
17 predetermined breaking point
18 tooth
19 region
20 openings

1.-34. (canceled)

35. A method for producing a covering device for a bone defect site (2), which has a top piece (4) and at least one fixing means (5) for the top piece (4), consisting of the following method steps:
Recording of a first dataset, which represents the affected bone detect site (2) in the actual state,
Comparison of the first dataset with a second dataset, which represents the actual state of a regenerated bone at the bone defect site (2),
Use of the first dataset and of the second the dataset for planning the top piece (4),
which is suitable to at least partially cover the bone defect site (2), wherein the top piece (4) has a side (11) facing away from the bone defect site (2) and a side (10) facing the bone defect site (2), and the top piece (4) is able to be fixed by at least one fixing means (5) within the bone defect site (2),
or
which is suited to at least partially cover the bone defect site (2) and in addition to at least partially cover a healthy bone (6) adjoining the bone defect site (2), wherein the top piece (4) has a side facing away from the healthy bone (6) and a side (11) facing away from the bone defect site (2) and a side facing the healthy bone (6) and a side (10) facing the bone defect site (2), and the top piece (4) is able to be fixed by at least one fixing means (5) within the bone defect site (2) and/or is able to be fixed to the healthy bone (6) by means of at least one fixing means (5),
Implementation of the planning of the top piece (4) into a planning dataset and
Delivery of the planning dataset to a manufacturing method, in particular to a computer-controlled manufacturing method, wherein during and/or after the manufacture of the top piece (4) at least one fixing means (5) suitable for fixing the top piece (4) within the bone defect site (2) or on the healthy bone (6) adjoining the bone defect site (2) is detachably connected to the top piece (4) by means of at least one connection (12) arranged between the fixing means (5) and the top piece (4), wherein the detaching of at least one connection (12) is irreversible and at least one connection (12) is a predetermined breaking point.

36. The method according to claim 35, characterized in that during at least one preceding method step for at least one fixing means (5) a fixing means position, adapted specifically to the use of this fixing means (5), which enables a fixing of the top piece (4) within the bone defect site (2) or on the healthy bone (6), is predetermined, in order to obtain, through the manufacturing method, a top piece (4) on which at least one fixing means (5) is detachably connected to the top piece (4) in its fixing means position.

37. The method, according to claim 36, characterized in that in the manufacturing method at at least one fixing means position, adapted for a fixing means (5), a guide is arranged for a tool, by which the creation of a bore, suitable for the fixing means (5), within the bone defect site (2) or on the healthy bone (6), is made possible.

38. The method according to claim 35, characterized in that the side (10) facing the bone defect or the side (11), facing away from the bone defect (11), of the top piece (4) formed in the manufacturing method, corresponds at least partially to the shape of the regenerated bone in the target state.

39. The method according to claim 35, characterized in that the second dataset arises through calculation or was recorded at a time at which the bone was still a healthy bone (6) at the bone defect site (2) which is now to be regenerated.

40. The method according to claim 35, characterized in that the recording of the first dataset represents the affected bone defect site (2) in its three-dimensionality and/or the recording of the second data set represents the shape of the still healthy bone (6) in its three-dimensionality.

41. The method according to claim 35, characterized in that the recording of the first dataset and/or the recording of the second dataset takes place by at least one imaging method.

42. The method according to claim 35, characterized in that the recording of the first dataset and/or the recording of the second dataset takes place by at least one method which enables a three-dimensional representation of a bone.

43. The method according to claim 35, characterized in that in the manufacturing method the top piece (4) is formed by means of 3D printing and/or by means of milling.

44. The method according to claim 35, characterized in that during the manufacture of the top piece (4) at least one fastening device for at least one implant which is to be set and/or at least one predetermined breaking point (17) and/or at least one positioning means (15) are arranged on the top piece (4).

45. The method according to claim 44, characterized in that at least one fastening device is exposed by removing a portion of the top piece (4) which before the removing is connected to the remaining portion of the top piece (4) by means of at least one predetermined breaking point (17).

46. The method, according to claim 44, characterized in that at least one positioning means (15), arranged on the top piece (4) during the manufacture of the top piece (4), serves for positioning the top piece (4) in an insertion position.

47. The method, according to one of claim 44, characterized in that between the top piece (4) and at least one positioning means (15) at least one predetermined breaking point is arranged and/or that between the top piece (4) and at least one fixing means (5) arranged on the top piece (4), at least one predetermined breaking point (13) is arranged.

48. The method according to claim 35, characterized in that the top piece (4) and/or at least one fixing means (5) and/or at least one positioning means (15) are formed from a dimensionally stable material.

49. A covering device (1) for a bone defect site (2),
with a top piece (4),
by which the bone defect site (2) is able to be at least partially covered, wherein the top piece (4) has a side (11) facing away from the bone defect site (2) and a side (10) facing the bone defect site (2),
or
by which the bone defect site (2) is able to be at least partially covered and in addition a healthy bone (6), adjoining the bone defect site (2) is able to be at least partially covered, wherein the top piece (4) has a side facing away from the healthy bone (6) and a side (11) facing away from the bone defect site (2) and a side facing the healthy bone (6) and a side (10) facing the bone defect site (2),
with at least one fixing means (5) for fixing the top piece (4) within the bone defect site (2) and/or with at least one fixing means (5) for fixing the top piece (4) on the healthy bone (6) adjoining the bone defect site (2),
characterized in that
on the top piece (4) at least one fixing means (5), suitable for fixing the top piece (4) within the bone defect site (2) or on the healthy bone (6) adjoining the bone defect site (2), is detachably arranged by means of at least one connection (12) arranged between the fixing means (5) and the top piece (4), wherein the detaching of at least one connection (12) is irreversible and at least one connection (12) is a predetermined breaking point (13).

50. The covering device (1), according to claim 49, characterized in that on the top piece (4) for a fixing means (5) a fixing means position, adapted specifically to the use of this fixing means (5), is present, which enables a fixing of the top piece (4) within the bone defect site (2) or on the healthy bone (6), or on the top piece (4) for at least one fixing means (5) at least one fixing means position, adapted specifically to the use of at least one fixing means (5) is present, which enables a fixing of the top piece (4) within the bone defect site (2) or on the healthy bone (6), wherein at least one fixing means (5) is detachably connected to the top piece (4) in its fixing means position which is adapted specifically to the use of this fixing means (5).

51. The covering device (1), according to claim 49, characterized in that the top piece (4) has at least one predetermined breaking point (17).

52. The covering device (1), according to claim 49, characterized in that at least one fixing means (5) projects over
the side (11) facing away from the bone defect site (2) or the side of the top piece (4) facing away from the healthy bone (6)
and/or
the side (10) facing the bone defect site (2) or the side of the top piece (4) facing the healthy bone (6).

53. The covering device (1), according to claim 49, characterized in that the top piece (4) has a guide for a tool at at least one fixing means position adapted for a fixing means (5).

54. The covering device (1), according to one of claim 49, characterized in that the top piece (4) consists of a dimensionally stable material.

55. The covering device (1), according to claim 49, characterized in that the side (10) facing the bone defect, or the side (11) of the top piece (4) facing away from the bone defect, corresponds at least partially to the shape of a regenerated bone at the bone defect site (2).

56. The covering device (1), according to claim 49, characterized in that the top piece (4) has at least one opening.

57. The covering device (1), according to claim 49, characterized in that the top piece (4) has at least one fastening device for at least one implant which is to be set.

58. The covering device (1), according to claim 57, characterized in that at least one fastening device is at least partially covered by a portion of the top piece (4), which is connected to the remaining portion of the top piece (4) by means of at least one predetermined breaking point.

59. The covering device (1), according to claim 49, characterized in that for positioning the top piece (4) on a healthy bone (6) adjoining the bone defect site (2), at least one positioning means (15) is arranged on the top piece (4), which positioning means has a side (16) facing away from the healthy bone and a side facing the healthy bone (6) and at least partially corresponding therewith.

60. The covering device (1), according to claim 59, characterized in that at least one predetermined breaking point is arranged between the top piece (4) and at least one positioning means (15).

61. The covering device (1), according to claim 49, characterized in that the covering device (1) is produced in accordance with the method for producing a covering device for a bone defect site (2) according to claim 35.
